(19) United States
(12) Patent Application Publication
(10) Pub. No.: US 2026/0357174 A1
(43) Pub. Date: Nov. 12, 2026

(54) MODULAR MICROFLUIDIC BENTHIC MICROBIAL FUEL CELL SYSTEM AND METHOD THEREOF

(71) Applicant: The United States of America, as Represented by the Secretary of the Navy, Arlington, VA (US)

(72) Inventors: Terak Benjamin Hornik, Monterey, CA (US); Emil P. Kartalov, Pacific Grove, CA (US); Angelica Sarmiento, San Diego, CA (US); Yolanda Meriah Arias-Thode, San Diego, CA (US)

(21) Appl. No.: 19/203,637

(22) Filed: May 9, 2025

(51) Int. Cl. C12M 3/06; B33Y 80/00; C12M 1/00; C12M 1/12

(52) U.S. Cl. CPC C12M 23/16; B33Y 80/00; C12M 23/06; C12M 23/34; C12M 23/40; C12M 29/00

(57) ABSTRACT

A microfluidic benthic microbial fuel cell (MBMFC) comprising: a microbial medium; microbial consortia; one or more microfluidic wedges comprising multiple microbial environment volumes, multiple channels, an input, an output, the multiple microbial environment volumes being in fluidic communications with the multiple channels, the input, and the output; and multiple electrical conductors coupled to the multiple microbial environment volumes and coupled to an electrical output.

### STATEMENT OF GOVERNMENT INTEREST

The present disclosure was made in the performance of official duties by one or more employees of the Department of the Navy, and thus, embodiments herein may be manufactured, used or licensed by or for the Government of the United States of America for governmental purposes without the payment of any royalties thereon or therefor.

### FIELD OF USE

The present disclosure relates, in general, to a system and method of harnessing energy from microorganisms. More specifically, the present disclosure relates to a modular microfluidic benthic microbial fuel cell.

### BACKGROUND

Generally, microbial fuel cells (MFC) generate electricity through the interaction between microorganisms and electrodes. The basic working principle involves the oxidation of organic matter by microorganisms at the anode, releasing electrons and protons. An anode then absorbs the electrons, which flow to the cathode by an external electrical circuit.

One of the primary challenges of MFCs is their relatively low power output compared to conventional energy sources. This limitation arises from several factors, including inefficient electron transfer mechanisms, suboptimal electrode materials and geometry, limitations in microbial metabolic activity, and electron quenching (electron being accepted by another acceptor).

Current capturing electrodes (anodes) are inefficient at harnessing electricity from electroactive microorganisms. This may be because the outer layers of most microbial species are composed of non-conductive lipid membranes, peptidoglycans, and lipopolysaccharides. The non-conductive lipid membrane, peptidoglycans, and lipopolysaccharides outer layers hinder direct electron transfer to the anode, therefore requiring a mediator to assist in transferring the electrons to the anode before electron losses occur due to neutralization or electron quenching.

Typical MFC systems improve power output by optimizing or improving the MFC design, which may involve increases in surface area or volume which increases microbial populations. However, scale up can increase challenges such as maintaining uniformity in the electrode architecture, efficient mass transfer of substrates and products, and problems with managing the increased reactor volumes, all the while not addressing electron transfer inefficiencies. Scaling up an MCF system comes with additional challenges that include manufacturing, industrial, and economics.

Therefore, what is needed is a cost-effective MFC that promotes microbial stability, efficient electron transfer, consistent and reliable power output, reparability, manufacturability, robustness of design, and scalability.

The background section is provided to reveal information believed by the applicant to be of possible relevance to the present technology. No admission is necessarily intended, nor should be construed, that any of the preceding information constitutes prior art against the present technology.

### SUMMARY

To minimize the limitations in the prior art, and to minimize other limitations that will become apparent upon reading and understanding the present specification, the present disclosure discloses a new and useful modular microfluidic benthic microbial fuel cell system and method thereof.

The following presents a simplified overview of the example embodiments in order to provide a basic understanding of some embodiments of the example embodiments. This overview is not an extensive overview of the example embodiments. It is intended to neither identify key or critical elements of the example embodiments nor delineate the scope of the appended claims. Its sole purpose is to present some concepts of the example embodiments in a simplified form as a prelude to the more detailed description that is presented herein below. It is to be understood that both the following general description and the following detailed description are exemplary and explanatory only and are not restrictive.

The problem of low electrical power generation per unit volume of microfluidic benthic microbial fuel cells (MBMFCs) or MFCs may be solved by increasing the volume and/or decreasing the distance between the microbes and the capturing electrodes. This may be achieved using a microfluidic fractal branch channel with embedded distributed anode wiring.

3D printed microfluidic fractal branch channels, geometric-shaped lattice environments, random/statistically distributed, and spicule lattice channels may contribute to the improvement of the harnessing of electrons from benthic microbial fuel cell systems.

In one embodiment, optimizing volumetric coverage and distance to electron capture point due to CNF wiring. The size and volume of a microbial environment may be optimally designed and otherwise properly designed to support the formation of a biofilm that may improve the ecological stability and the electrogenic effect of the microorganisms living within the microfluidic fractal branch channel, geometric-shaped lattice environments, spicule lattice channels, and/or housed by a MFC. Additional microbial environment support factors may include surface roughness, surface area, volume, material, porosity, and any other parameter that may affect biofilm development or ecological development. Electron production may be improved by controlling the inflow of nutrients, and/or the removal of waste byproducts may be controlled to improve microorganism health and electron production. The environments intended for microorganisms may be 3D printed utilizing additive manufacturing techniques to create modular geometric components units such as (but not limited to) modular wedges. The wedges may be designed as a part of a larger unit, such as (but not limited to) a tray, ring, plate, platter, or any other planar, stackable, or otherwise designed to facilitate assembly geometrically shaped unit that may facilitate formation of an anode and/or form an anode. The modular geometric components may be designed to accept a fluid flow from a single microbial medium inflow while being fluidically isolated downstream from any additional modular geometric components and be electrically coupled to aggregate any electron collection. The modular geometric assembly’s components may also include conductors that provide a method for the electrons to be utilized in electrical and/or electronic circuits.

Because the modular geometric assembly’s components can be fluidically isolated downstream from any other module, should they become individually clogged, partially clogged, or otherwise possess reduced flow, units that suffer from reduced flow should not significantly impact the functional capacity of other units. Additionally, a locus with a clog or with reduced flow may have a reverse flow applied to remove any obstruction.

Cyclically applying a reverse flow may prevent clogs or obstructions from degrading flow. A regulator and/or load and a cathode may also be electrically attached to a formed single connected anode to form a completed MFC or MBMFC.

A modular microfluidic benthic microbial fuel cell system in its simplest form may be a cathode in an oxygenated environment, anode in a reducing environment, and associated electrical and/or electronic components. The cathode and associated electrical and/or electronic components may be configured to operate in a water column with the anode under anaerobic or fermentative conditions. In one embodiment the anode may be assembled inside an array of modular geometric components that may be connected, attached, or arranged in a planar or stackable orientation, such as a tray, wafer, plate, platter, or ring. The assembled modular geometric components may be stacked on other assembled modular geometric components and the anodes in each module may be electrically coupled to form a single connected anode.

One embodiment of a microfluidic benthic microbial fuel cell (MBMFC) may comprise: a microbial medium; a microbial consortia; multiple microfluidic wedges arranged on a plane to form a tray, each of the microfluidic wedges may comprise multiple microbial environment volumes, multiple channels, an input, an output, the multiple microbial environment volumes being in fluidic communications with the multiple channels, the input, and the output; and multiple electrical conductors coupled to the multiple microbial environment volumes and coupled to an electrical output. Wherein the microbial medium flows through the input, the multiple channels, the multiple microbial environment volumes, and the output; wherein the microbial consortia live in the multiple microbial environment volumes; and wherein the multiple microbial environment volumes may be configured to capture electrons. Wherein the dimensions of the multiple microbial environment volume range from 1 micro-meter cubed (µm3) to 10 cubic meters (m3); and the multiple microbial environment volumes receive a nutrient import and waste export through the flow of the microbial medium. Wherein the multiple microbial environment volumes may be configured to retain a biofilm layer comprising the microbial consortia and an extracellular polymeric substance. The MBMFC may further comprise an electrical regulator or a load; the electrical regulator or load being electrically coupled to the output of the microfluidic wedges. The MBMFC may further comprise a cathode electrically coupled to the electrical regulator or load. The MBMFC may further comprise multiple microfluidic trays stacked to form an anode; and the multiple electrical conductor outputs being coupled to the electrical regulator or load. Wherein the multiple microbial environment volumes, the multiple channels, the input, the output may be made from carbon or a conductive matrix. Wherein the carbon may be carbon nanotubes. Wherein the carbon may be graphene or carbon nanofibers. Wherein the carbon may be carbon nanotubes, carbon nanofibers, and graphene. Wherein the multiple electrical conductors may be biocompatible conductive conductors selected from the group consisting of: stainless steel, chrome, titanium, tantalum, niobium, and gold.

One embodiment of a microfluidic benthic microbial fuel cell (MBMFC) may be produced by the process of: providing a MBMFC microfluidic design comprising multiple microbial environment volumes, multiple channels, an input, an output, the multiple microbial environment volumes being in fluidic communications with the multiple channels, the input, and the output; providing multiple carbon nanofibers; providing multiple electrical conductors; 3-dimensional printing multiple layers, a single layer at a time, of the MBMFC microfluidic design by extrusion of the multiple carbon nanofibers; self-assembling, by the multiple electrical conductors, wire conductors electrically coupled to the multiple microbial environment volumes and an electrical output; and coupling electrical outputs to an electrical regulator or a load. The MBMFC process may further comprise providing a microbial medium and providing a microbial consortia, wherein the microbial consortia may be a bacteria. Wherein the dimensions of the multiple microbial environment volume range from 1 cubic micro-meter (µm3) to 10 cubic meters (m3); and wherein the multiple microbial environment volumes receive a nutrient import through the flow of the microbial medium. Wherein the multiple microbial environment volumes may be configured to export a waste through the flow of the microbial medium. The MBMFC process may further comprise providing a cathode electrically coupled to the electrical regulator or load. The MBMFC process may further comprise: combing multiple microfluidic wedges to form a tray; stacking multiple trays to form an anode; and electrically coupling multiple electrical outputs of the multiple microfluidic wedges to the electrical regulator or load.

One embodiment of a method of harnessing electrogenic power by a microfluidic benthic microbial fuel cell (MBMFCs) comprising: providing a MBMFC cell comprising microbial environment volumes, channels, an input, an output, the microbial environment volumes being in fluidic communications with the channels, the input, and the output; providing electrical conductors coupled to the microbial environment volumes and coupled to the electrical outputs; coupling the electrical outputs to a tray output; coupling the tray outputs to an electrical regulator or a load; providing a microbial consortia, wherein the microbial consortia releases electrons and wherein the released electrons may be collected by the MBMFC cell; and providing a microbial medium, wherein the microbial medium flows through the input, the multiple microbial environment volumes, the multiple channels, and the output. The method of harnessing electrogenic power by an MBMFCs may further comprise providing a cathode; and an electrical regulator or a load.

It is an object to overcome the limitations of the prior art.

These, as well as other components, steps, features, objects, benefits, and advantages, will now become clear from a review of the following detailed description of illustrative embodiments, the accompanying drawings, and the claims.

### BRIEF DESCRIPTION OF THE DRAWINGS

The drawings are of illustrative embodiments. They do not illustrate all embodiments. Other embodiments may be used in addition or instead. Details which may be apparent or unnecessary may be omitted to save space or for more effective illustration. Some embodiments may be practiced with additional components or steps and/or without all of the components or steps which are illustrated. When the same numeral appears in different drawings, it refers to the same or like components or steps.

FIG. 1 is an illustration of a modular microfluidic benthic microbial fuel cell (MBMFC).

FIG. 2 is one embodiment of a cross-sectional view of a fractal branch channel showing the electrogenic process.

FIG. 3A is an alternate embodiment of a printed hexagon lattice channel model.

FIG. 3B is an alternate embodiment of a printed carbon spicule lattice model.

FIG. 4 is an illustration of an individual wedge of a modular microfluidic benthic microbial fuel cell (MBMFC) having a single-layered fractal branch channel.

FIG. 5 is an illustration of an individual wedge of a modular microfluidic benthic microbial fuel cell (MBMFC) having three fractal branch channel layers.

FIG. 6 is an illustration of an individual wedge of a modular microfluidic benthic microbial fuel cell (MBMFC) having three fractal branch channel layers with an increase in fractal channel length.

FIG. 7 is one embodiment of a cylindrically assembled modular microfluidic benthic microbial fuel cell (MBMFC) having seven stacked layers and six radially arranged wedge modules per layer.

FIG. 8 is one embodiment of a microfluidic benthic microbial fuel cell (MBMFC) having an external enclosure.

FIG. 9 is a block flow diagram showing the steps to produce a microfluidic benthic microbial fuel cell (MBMFC).

FIG. 10 is a block flow diagram of one method of harnessing electrogenic power by a microfluidic benthic microbial fuel cell (MBMFC).

FIG. 11A is an illustration of one method of wiring a microfluidic benthic microbial fuel cell (MBMFC).

FIG. 11B is an illustration of one method of wiring together individual modules of an MBMFC.

### DETAILED DESCRIPTION OF THE ILLUSTRATIVE EMBODIMENTS

In the following detailed description of various embodiments of the present disclosure, numerous specific details are set forth in order to provide a thorough understanding of various aspects of one or more embodiments of the present disclosure. However, one or more embodiments of the present disclosure may be practiced without some or all of these specific details. In other instances, well-known methods, procedures, and/or components have not been described in detail so as not to unnecessarily obscure aspects of embodiments of the present disclosure.

While multiple embodiments are disclosed, still other embodiments of the devices, systems, and methods of the present disclosure will become apparent to those skilled in the art from the following detailed description, which shows and describes illustrative embodiments of the devices, systems, and methods of the present disclosure. As will be realized, the devices, systems, and methods of the present disclosure are capable of modifications in various obvious aspects, all without departing from the spirit and scope of the present disclosure. Accordingly, the screenshot figures, and the detailed descriptions thereof, are to be regarded as illustrative in nature and not restrictive. Also, the reference or non-reference to a particular embodiment of the devices, systems, and methods of the present disclosure shall not be interpreted to limit the scope of the present disclosure.

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that may be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all embodiments of this application including, but not limited to, steps in disclosed methods. Thus, if there are a variety of additional steps that may be performed it is understood that each of these additional steps may be performed with any specific embodiment or combination of embodiments of the disclosed methods.

The present methods and systems may be understood more readily by reference to the following detailed description of preferred embodiments and the examples included therein and to the Figures and their previous and following description.

In the following description, certain terminology is used to describe certain features of one or more embodiments. For purposes of the specification, unless otherwise specified, the term “substantially” refers to the complete or nearly complete extent or degree of an action, characteristic, property, state, structure, item, or result. For example, in one embodiment, an object that is “substantially” located within a housing would mean that the object is either completely within a housing or nearly completely within a housing. The exact allowable degree of deviation from absolute completeness may in some cases depend on the specific context. However, generally speaking, the nearness of completion will be so as to have the same overall result as if absolute and total completion were obtained. The use of “substantially” is also equally applicable when used in a negative connotation to refer to the complete or near complete lack of an action, characteristic, property, state, structure, item, or result.

As used herein, the terms “approximately” and “about” generally refer to a deviance of within 5% of the indicated number or range of numbers. In one embodiment, the term “approximately” and “about”, may refer to a deviance of between 0.001-10% from the indicated number or range of numbers.

Various embodiments are now described with reference to the drawings. In the following description, for purposes of explanation, numerous specific details are set forth in order to provide a thorough understanding of one or more embodiments. It may be evident, however, that the various embodiments may be practiced without these specific details. In other instances, well-known structures and devices are shown in block diagram form to facilitate describing these embodiments.

As used herein, the term “benthic” refers to being associated with or occurring on the bottom of a body of water.

As used herein, the term “electrogenic” refers to the production of electrical activity in living tissue.

As used herein, the term “microbial medium,” “microbiological culture medium,” or “bacterial culture medium” refers to a substance that supports the growth of microorganisms.

As used herein, the term “microfluidic” or “milli-fluidic” refers to the behavior of fluids through networks containing micro-scale or milli-scale channels and the technology of manufacturing microminiaturized devices containing chambers and tunnels through which fluids flow or are confined.

Microfluidic benthic microbial fuel cells (MBMFCs) may provide microbial stability, rich nutrient supply, efficient microbial waste removal, electrode architecture, and/or may improve electron transfer. Taking advantage of biomimicry, an MBMFC may benefit from the general fluid dynamics possessed by Aplysina fistularis (sea sponge animal).

Flow in and out may be directed from the outside of the cylindrical structure towards the central axis and then out the top or bottom of the central chamber of the structure. Alternatively, fluid may flow in the opposite direction as with the sponge animal but could be in either direction or reversable with no change to the design of the module, the fabrication process, or the assembly process.

MBMFCs may have multiple microfluidic channels and/or microbial environmental volumes that may be interconnected to share nutrient-carrying flows and/or waste removal. The interconnections and improvements to conductivity of the MBMFCs may improve the harnessing and efficiency of harnessing electricity through decreased resistivity and/or additional electrical pathways.

The potential for increased power output in MBMFCs may be a promising aspect of this disclosure. Individual MBMFCs may generate low electrical power per unit volume, typically in the range of 5-20 milli-watt per cubic meter (mW/m³). However, this output power generation may be significantly increased by scaling the number of MBMFC modules, adjusting/introducing microfluidic fractal channels, and/or by increasing the power output density by decreasing the mean distance from the emitting bacterium to the capturing electrode, by controlling the size of the microbial environmental volume and/or microfluidic channels. This may lead to a substantial increase in power density. This potential for increased power density and output offers a hopeful future for the field of bioenergy.

3D printing, microfluidic fractal branch design, assembly level design, fragmentation, and modular design may allow for scalability of MBMFCs and may be a promising aspect of this disclosure. The individual fragments or modules may enable the creation of scalable MBMFCs and/or anodes. This scalability could significantly improve MBMFC power production. 3D printing may further improve manufacturing and/or production of MBMFCs in fragments or modular components, giving the designer control over the most relevant dimensions while allowing for increases efficiency. A modular designed MBMFC may have its power output scaled up or down by increasing or decreasing the proportion of the volume given over to internal channels, increasing or decreasing the number of modular units in the assembly, increasing or decreasing the number of fractal branch channel layers in a single wedge unit, increasing or decreasing the outer radius or outer perimeter of the wedge, increasing or decreasing the inner radius or inner perimeter of the wedge, and/or increasing or decreasing the number of wedges per disk. This may allow MBMFC to be tailored to a particular environment and/or use case. By making selections at the assembly level (e.g., choosing a larger or smaller number of modules) the process of customizing a MBMFC may be accomplished with no change to the actual fragments or modules, thus allowing for a single fixed fragment/module design to have an increased range of application. With a cylindrical assembly design this may be accomplished by increasing or decreasing the height of the cylinder by adding or removing layers of modules with no need to change other aspects of the design.

The groups of microorganisms that demonstrate the ability to transfer electrons from their cells to the electrodes are referred to as exoelectrogens. Exoelectrogens may be obtained from various environments, such as but not limited to wastewater, compost, manure, dirt, river or lake sediments, swamps, and marine ecosystems. However, there are many other organisms in the environment that have yet to be studied and may be important in our systems that may be contributing electrons.

MBMFC assemblies may allow seawater, containing electrogenic bacteria, to be artificially and/or naturally pumped into a central axial macro cavity formed by the assembly or directly into a network of microchannels. A pressure differential may drive flow from the inside to the outside (or the outside to the inside) of the overall cylindrical assembly in a radial direction. Flow occurs in each individual layer of microfluidic channels independently as they are all connected by a single inlet port to the inner axial cavity effectively removing the need for microfluidic-to-microfluidic interfaces between modules and simultaneously improving the robustness of the overall MBMFC to fluidic type failures. This type of arrangement may significantly reduce the number of single-point failures involved in microfluidics as each layer on each module can be thought of as physically independent in the downstream direction (or upstream if the flow was oriented from the outside to the inside of the cylinder). Additionally, this type of arrangement may significantly reduce the effect of a fluidic failure such as but not limited to a clog.

Flows may be driven from the tides, waves, currents, or other naturally occurring patterns of seawater flow with alternating directions, consistent and thus predictable directions, sufficient flow energy, and/or regular occurrence. This may be exacerbated via or geometrically imposed flow utilizing concepts such as but not limited to the reverse step effect or centripetal acceleration. Additionally, nutrients can be added into the system if desired. In a closed or partially closed fluidic loop nutrients and/or microbes may be recirculated.

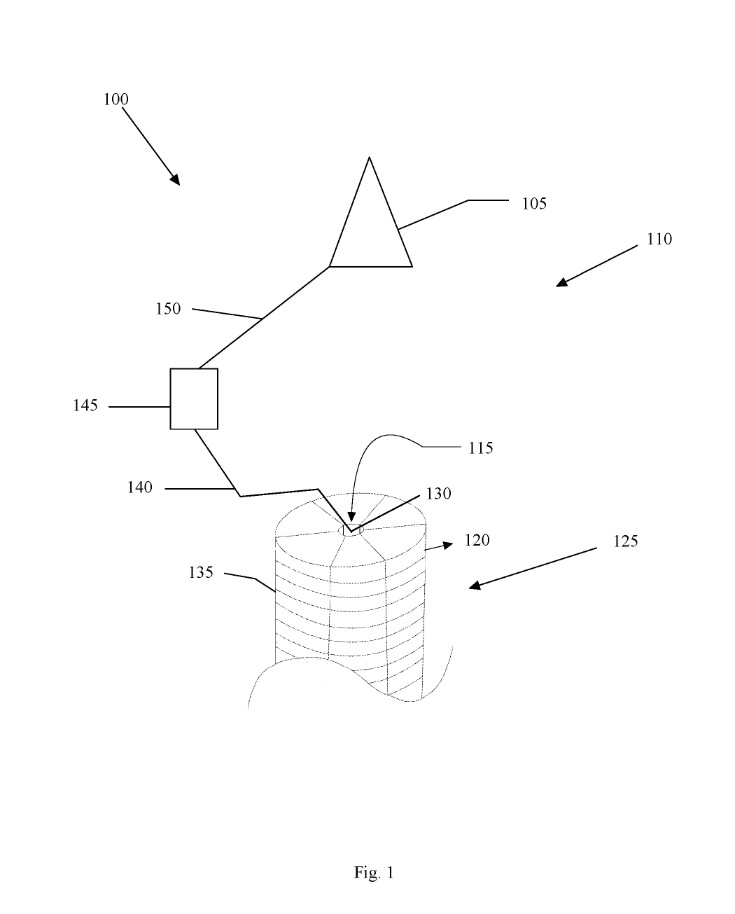

FIG. 1 is an illustration of a modular microfluidic benthic microbial fuel cell (MBMFC). A modular microfluidic benthic microbial fuel cell (MBMFC) 100 may include and/or interact with cathode 105, water column 110, microbial medium inflow 115, microbial medium outflow 120, anode 125, coupled electrical output 130, Microfluidic benthic microbial fuel cell (MBMFC) wedge 135, anode electrical input 140, electronic regulator 145, and regulator output 150.

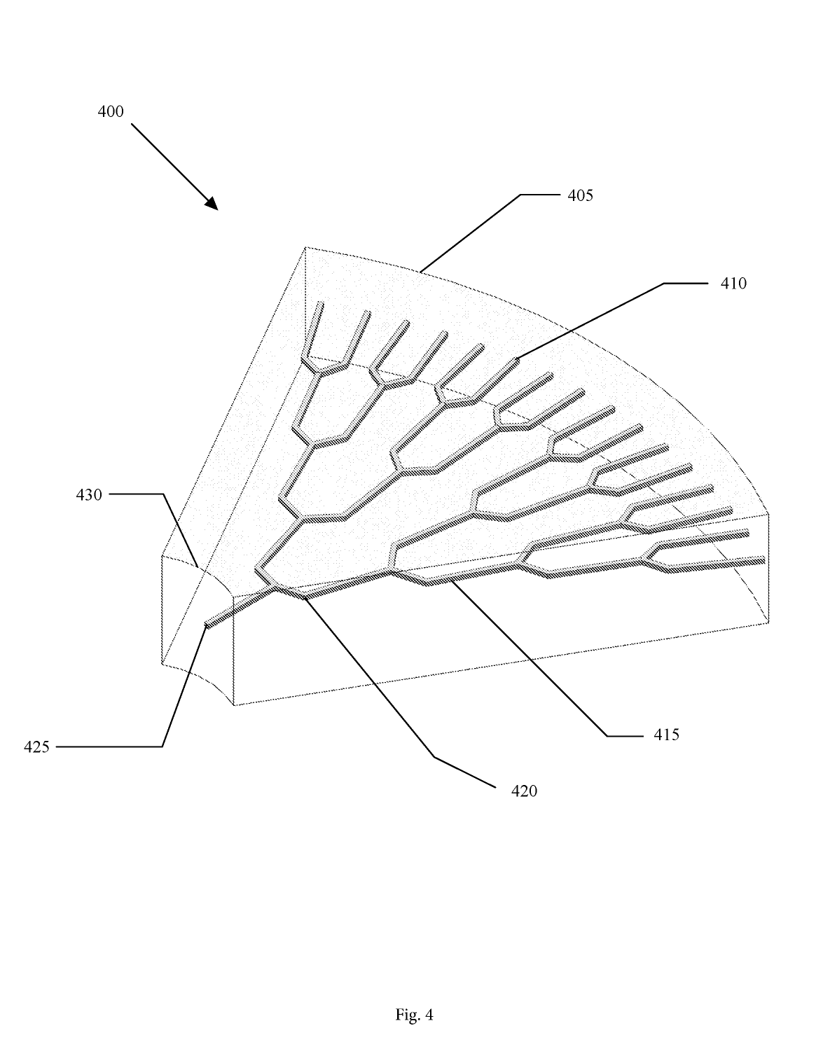

Anode 125 may be formed from or in multiple MBMFC wedge 135. The number of MBMFC wedge 135 may be added or removed based on the power output need and/or environmental conditions. MBMFC wedge 135 may include electrical output 130, and as shown in FIG. 4, fluid input 425 and one or more fluid outputs 410. Electrical output 130 may be coupled to the electrical output of multiple MBMFC wedge 135. Electrical output 130 may preferably be routed through microbial medium inflow 115 but may also be routed externally along the microbial medium outflow 120, though the body of the module, and/or through other electrical pathways. Nutrient-rich fluid may flow from water column 110 through microbial medium inflow 115 into each MBMFC wedge 135 through fluid input 425. Alternatively, nutrient-rich fluid may be directly added to fluid input 425 (as shown in FIG. 4), microbial medium inflow 115, and/or water column 110. Waste-carrying fluid may flow out of each MBMFC wedge 135 through output 410 and return to water column 110 through microbial medium outflow 120, or in the reverse direction. Alternately, nutrient-rich fluid flow from water column 110 may be directed to self-clean, clear a clog, clear a blockage through microbial medium outflow 120 and into each output 410 of MBMFC wedge 135 by changing the orientation of the flow. Waste-carrying fluid may flow out of each fragmented segmented input 425 and out through microbial medium inflow 115, returning to water column 110.

Electronic regulator 145 may comprise electrical input 140 and regulator output 150. Electronic regulator 145 may be an electrical load, a resistive load, a voltage regulator, a current regulator, an energy storage system, and/or directly connected to a system requiring power. Electronic regulator 145 may include adaptive software, electrical or galvanic isolators, maximum power point tracking, data collection, and/or adaptive energy harvesting controls (not shown). Electrical input 140 may be one or more electrical connections to one or more anodes and may supply current and voltage from anode 125 to electronic regulator 145 for use in an electrical or electronic system. Regulator output 150 may be a single electrical connection to cathode 105 or many parallel connections to a cathode bundle.

Water column 110 may be a microbial medium that provides nutrients for the growth of microorganisms. Water column 110 may also provide through flow to remove biological waste or other material needing removal. Water column 110 may also provide fluid flows from the tides, waves, currents, or other naturally occurring patterns of seawater flow with alternating and/or constant directions. This may be exacerbated via or geometrically imposed flow utilizing concepts such as but not limited to the reverse step effect or centripetal acceleration. Additionally, nutrients can be added into water column 110 if desired.

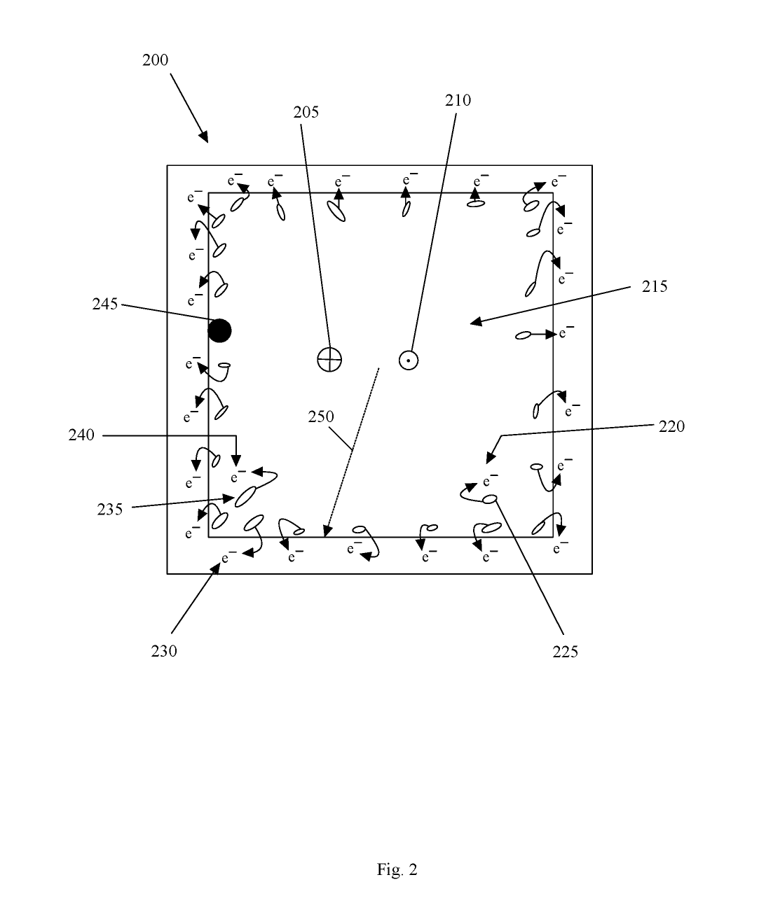

FIG. 2 is one embodiment of a cross-sectional view of a fractal branch channel. Fractal branch channel 200 may be, but should not be limited to, rectangular, circular, triangular, tear drop, or ovular-shaped. As shown in FIG. 2, fractal branch channel 200 may have inflow 205 and outflow 210 depending on the need of a user or requirements of MBMFC 100 these flow directions may be reversed.

Fractal branch channel 200 may be microfluidic and may have channel dimension 250 described by a set of dimensions such as but not limited to, a line segment, a length, and width for a rectangular channel, a radii for a circular channel, a base and height for a triangular channel, or a short and long axis for an oval. Channel dimension 250 may be a design parameter that may be chosen based on, but should not be limited to, fabrication tolerances, fabrication resolutions, materials, fluid properties, fluidic resistance, ability to clear and/or process, desired flowrate, deposition of CNF wiring, other wiring methods, aspect ratios, channel stability, surface area to volume ratios, variety of microorganisms (indigenous and/or introduced microorganisms), robustness to clogging, seeded bacteria, environmental requirements, architectural confinement, and/or probability of electron transfer by the anode. Channel dimension 250 may be limited by manufacturing technology and/or production limitations. Channel dimension 250 may be a design dimension that effects free volume 215. Free volume 215 may allow inflow 205 and outflow 210 of fluid from the microbial environment. A smaller channel cross-sectional size may increase the probability of electron 230 transfer to anode 125 due to decreasing the average distance from the emitted electron to the capturing electrode thereby decreasing the recombination probability as well as the energy needed to capture the electron. This decrease of the average distances may also be achieved by distributing the electrode wiring thought the free volume 215. Although larger channel dimension 250 may increase the surface area available for electron capture the probability of electron 230 being transferred to anode 125 may decrease due to an increase in electron loss probability from neutralization and/or electron quenching.

The inner surface area of the fractal branch channel 200 may allow for microorganisms 225 and 235 to attach, propagate, and/or develop a biofilm that may enhance the environment and assist in electron production. Microorganisms 225 and 235 may release electrons 220, 230, and 240 through biological processes. Electrons 220, 230, and 240 may transfer to anode 125. Electrons 220, 230, and 240 may also be lost to neutralization or electron quenching. This may be exacerbated by free volume 215 being too large, oxygen (or other terminal electron acceptor) getting into the system, inefficient waste removal, other microorganisms 225, and/or 235 contaminants.

Biocompatible conductor 245 preferably will readily conduct most electrons 220, 230, and 240 released by microorganisms 225 and 235. Biocompatible conductor 245 may be made from carbon, stainless steel, chrome, titanium, tantalum, niobium, and/or gold. It is preferable that materials may be biocompatible and marine quality conductors.

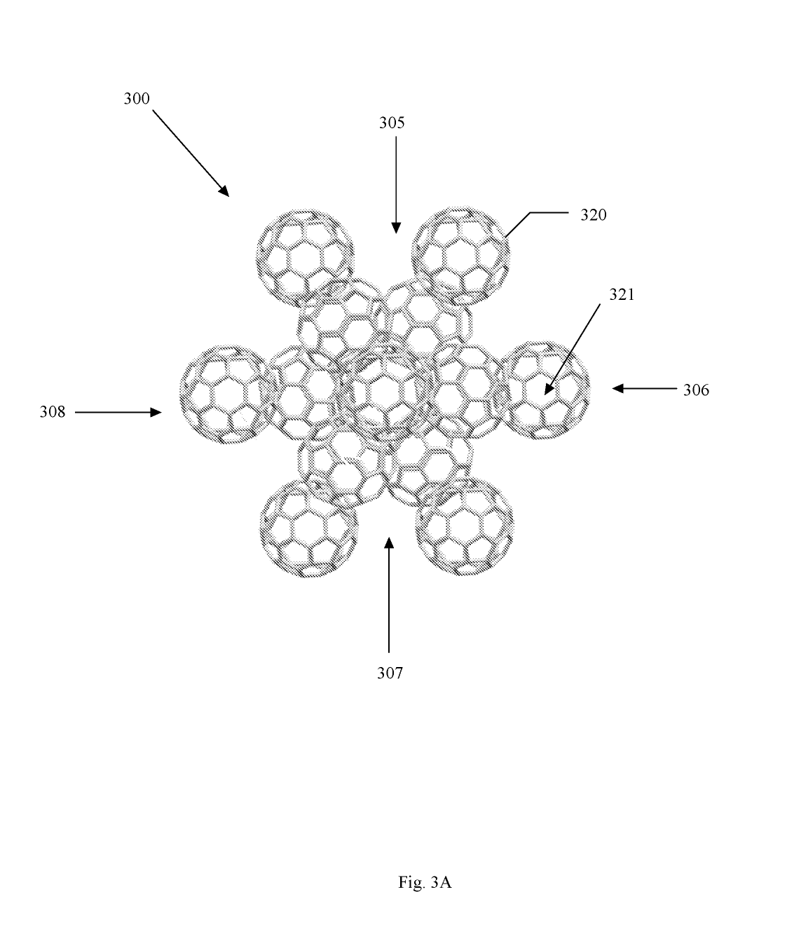

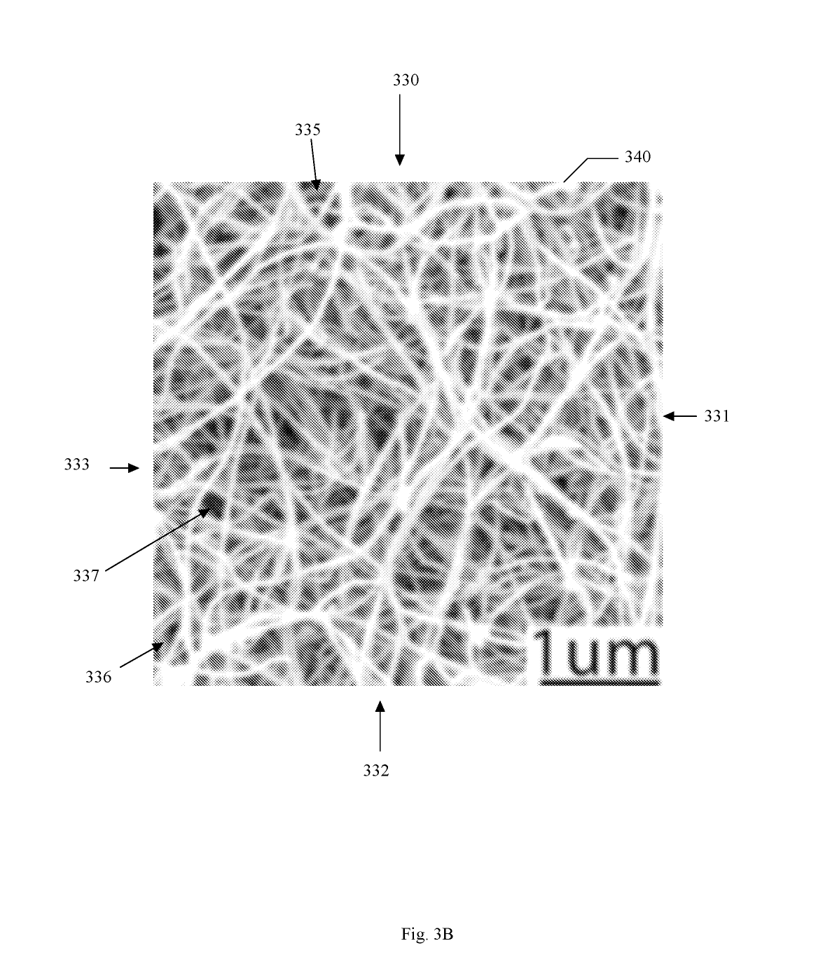

FIG. 3A is an alternate embodiment of a printed hexagon lattice channel model. Fractal branch channel 300 may be a lattice of multiple geometric-shaped microorganism environments 320. As shown in FIG. 3A, microorganism environment 320 may be a carefully designed hexagonal structure but should not be limited to only a hexagonal polygon. Microorganism environment 320 may include multiple free space 321 openings that may allow water column 110 fluid to flow in and out of microorganism environment 320. Microorganism environment 320 may be 3D printed, and the size of microorganism environment 320 may be limited by current printing technologies. The size and volume of microorganism environment 320 should be large enough for a single microorganism, but preferably large enough to support a biofilm produced by various microorganisms.

Electrodes (not shown) may be made from biocompatible materials such as but not limited to the following: titanium, carbon, carbon nanotubes, carbon nanofibers, graphene, and/or other electrically conductive biocompatible material.

Biocompatible conductor 245 (as shown in FIG. 2) may be constructed using self-assembling wires after fractal branch channel 300 has been formed into a 3D lattice. Self-assembling wires may consist of electrically polarized particles which self-assemble in response to an electric field. They can be used to create complex electrical devices, shape memories, and reconfigurable structures. The conductivity of self-assembling wires may be optimized between microorganism environments and the resistance of the wire can be changed in response to externally imposed boundary conditions. Alternatively, biocompatible conductor 245 (as shown in FIG. 2) may be constructed using chemical derivatization by active groups using standard coupling reactions, e.g., peptide bonding or opening a vinyl bond (polymerization). Alternatively biocompatible conductor 245 (as shown in FIG. 2) may also be constructed using deposition by flow and/or evaporation, wherein conductive microfibers mechanically enmesh and become stuck electrostatically, by Van der Waals forces, hydrophilic/hydrophobic interactions, and/or by other means.

Fractal branch channel 300 may not have a dedicated input and output, but may flow from direction 305, 306, 307, and 308. The flow may be caused by, but should not be limited to, currents, tides, waves, pumps, and/or other sources of pressure differential. Flow from naturally occurring sources, such as but not limited to, currents, tides, waves, or geometrically imposed flow (such as but not limited to the reverse step effect or centripetal acceleration) may allow anode 125 (as shown in FIG. 1) to harness electricity without the need for an external pump. Alternatively, the MFC may power an external pump, or it may be supplemented or replaced by externally available power.

Another embodiment may include an entire fragmented module formed by a structure similar to standard infill patterns commonly used in FDM printers, such as but not limited to gyroid infill. This embodiment may allow both the structure defining microbial environment and the anode to be simultaneously formed by the geometry which may be fabricated via 3D printing using a material such as carbon spiked PLA. This embodiment may allow for these to be fabricated as fragmented modules or as a single monolithically printed device.

In one embodiment, the printed carbon nanotube hexagon lattice structure may be 3D printed to form an anode that is not limited to any specific volume or geometry. A single anode may be 3D printed using a carbon nanotube hexagon lattice. In this embodiment, the fluid may flow through the entire structure and the entire structure may be used as the environment for microbes, overall physical structure, and anode simultaneously.

In one embodiment the MFC’s (or MBMFC’s) anode, microbial environment, and physical structure may be simultaneously formed by a 3D printed porous geometry made of a conductive material. This porosity may be achieved, but should not be limited to being achieved, via typical infill patterns used with FDM 3D printing. This conductive material may be carbon spiked PLA or any other conductive, biocompatible, and 3D printable material. It may be advantageous for the porosity of the geometry to be isotropic. Alternatively, it may be advantageous to have a directional bias to control flow movements. Surface tension may be used in conjunction with or in place of other methods of distributing the fluid containing microorganisms thought the volume of the porous geometry. This embodiment may provide any of the following benefits: increases surface area, increased surface area per unit volume, increases support of biofilm formation, many fluidic pathways (no fluidic single point failures), resistance to clogging, multi directional fluid dynamics, potential significant decrease in production/equipment costs, increased degassing ability (for waste products), increased accessibility (due to compatibility with FDM 3D printing), a decreased need for manual or automated post processing after 3D printing, scalability at both the scale of the internal porosity and the external geometry, and compatible with modular design while still allowing for a non-modular monolithic design.

FIG. 3B is an alternate embodiment of a printed carbon spicule lattice model. Fractal branch channel 340 may be printed as 3-dimensional lattice of multiple spicules that form microorganism environments 336 and 337. As shown in FIG. 3B, microorganism environments 336 and 337 may be a random design and or a previously geometrically defined shape that may be a printed lattice of spicules. Microorganism environments 336 and 337 may include multiple free space 335 openings or a single connected free space 335 that may allow water column 110 fluid to flow in and out of microorganism environments 336 and 337. Microorganism environments 336 and 337 may be deposited or fabricated via 3D printing, via post-processing methods, and/or using other approaches. Current printing technologies and/or fabrication methods may limit the size of microorganism environments 336 and 337 and/or other aspects of the design. The size and volume of microorganism environments 336 and 337 should be large enough for a single microorganism, but preferably large enough to support a biofilm produced by multiple microorganisms.

Fractal branch channel 340 may be printed from common and/or uncommon 3D printing materials. Fractal branch channel 340 may be directly printed or further coated, impregnated, deposited, assembled, self-assembled, lined, and/or otherwise augmented so as to produce simultaneous conductivity and flow. This post print processing may be done using carbon, carbon nanotubes, carbon nanofibers, graphene, titanium fragments, gold fragments, titanium particles, gold particles, and/or other forms of carbon, but should not be limited to these materials.

Biocompatible conductor 245 may be constructed using self-assembling wires after a fractal branch channel has been formed into a 3D lattice. Self-assembling wires may consist of, but should not be limited to, electrically polarizable particles which self-assemble in response to an electric field. They can be used to create complex electrical devices, shape memories, and reconfigurable structures. The conductivity of self-assembling wires may be optimized between nodes, and the resistance of the wire can be changed in response to externally imposed boundary conditions and/or other stimuli.

Fractal branch channel 340 may not have a dedicated input and output, but may have flow from directions 330, 331, 332, and 333.

Another embodiment may include an entire fragmented module formed by a structure similar to standard infill patterns commonly used in FDM printers. This embodiment may allow both the structure defining microbial environment and the anode to be simultaneously formed by the geometry which may be fabricated with a material such as, but not limited to, carbon spiked PLA.

FIG. 4 is an illustration of an individual wedge of a modular microfluidic benthic microbial fuel cell (MBMFC) having a single-layered fractal branch channel. Microfluidic benthic microbial fuel cell (MBMFC) wedge 400 may include fluid input 425, fluid output 410, fractal branch channels 420, 415, inner inflow arc 430, and outer output arc 405. MBMFC wedge 400 may be one of many units that form one or more stackable layers (as shown in FIG. 1) in a MBMFC containing anode 125.

MBMFC wedge 400 may preferably be standardized in design, shape, pattern, and size, which may allow for modularity, replaceability, ease of manufacturing, ease of assembly, scalability, and/or lower costs. Lower costs may include, but should not be limited to, lower production costs, lower repair/maintenance costs, lower human/labor costs, and/or other possible cost decreases.

MBMFC wedge 400 design may allow for in-situ replacement of individual MBMFC wedge 400. In-situ replacement may be aided by a radial guide rail and/or other guiding mechanisms. 3D printed MFCs as well as 3D printed MBMFCs in particular may be limited by the dimensions of the available printing volume that the chosen 3D printer(s) may be capable of using. This limitation may often prevent a 3D printed MFC or MBMFC to be designed and manufactured monolithically. Alternatives to the generally more desirable monolithic approach that may bypass this constraint include but should not be limited to the following: modules, fragments, and/or using other piecewise techniques. One embodiment of such a module may be the MBMFC wedge 400 shown in FIG. 4. As real estate in the print volume may be highly valuable, it may be advantageous to optimize the portion of the available space used when printing. MBMFC wedge 400 geometry may have features that lend itself to a nestable design on the bed of a 3D printer that may offer increased production efficiency, decrease human involvement, potential decrease in material usage, and/or higher yields. Fractal branch channels 420 and 415 may be circular, rectangular, triangular, oval-shaped, tear-drop shaped, or possess other cross-section designs to form the internals of a MBMFC wedge 400. Alternatively, fractal branch channels 420 and 415 may be a lattice of multiple geometric-shaped microorganism environments 320 (as shown in FIG. 3A). Alternatively, fractal branch channels 420 and 415 may be a lattice of multiple spicules that form microorganism environments 336 and 337 (as shown in FIG. 3B).

Designing fractal branch channels 420 and 415 without sharp changes in direction may minimize the resistance of the layers inside of MBMFC wedge 400, further decreasing the required pressure input to create equivalent flow and potentially decreasing the back work ratio. Smooth continuous fractal branch channels 420 and 415 provide a streamlined and naturalistic appearance of fractal branch channels 420 and 415 and may contribute to the reduction of fluidic resistance. The geometry of fluid output 410 may preferably be designed to minimize head loss for the internal flow. The geometry of fluid input 425, fluid output 410, and/or fractal branch channels 420 and 415 may be designed for easier processing, wiring, and robust channel spacing.

Fractal branch channel 415 and 420 may be constructed within MBMFC wedge 400 and augmented with carbon, carbon nanotubes, carbon nanofibers, graphene and/or other conductive materials. Electrical conductivity may be critical, as it allows the electrons to be captured and thus allows current to flow and electrical power to be extracted. Biocompatible conductors ensure that the anode material may not be toxic to the bacteria and allow the microorganisms to be adjacent to a readily available terminal electron acceptor to capture the electrons. The CNF/conductive material matrix porosity allows the bacteria to move about fractal branch channel 420 and 415. Porosity also allows for fluidic throughput, which may be necessary to get microorganisms inside the microchannels, cycle them out if desired, remove waste products, and/or provide nutrients. Porosity also allows for the bacteria to be effectively inside the anode 125 (as shown in FIG. 1), thus further decreasing the distance from electron emission to electron capture.

Fractal branch channels 420 and 415 may be resized or their dimensions altered based on physical traits of the fluid and or microorganisms that may aid in preventing bottlenecking.

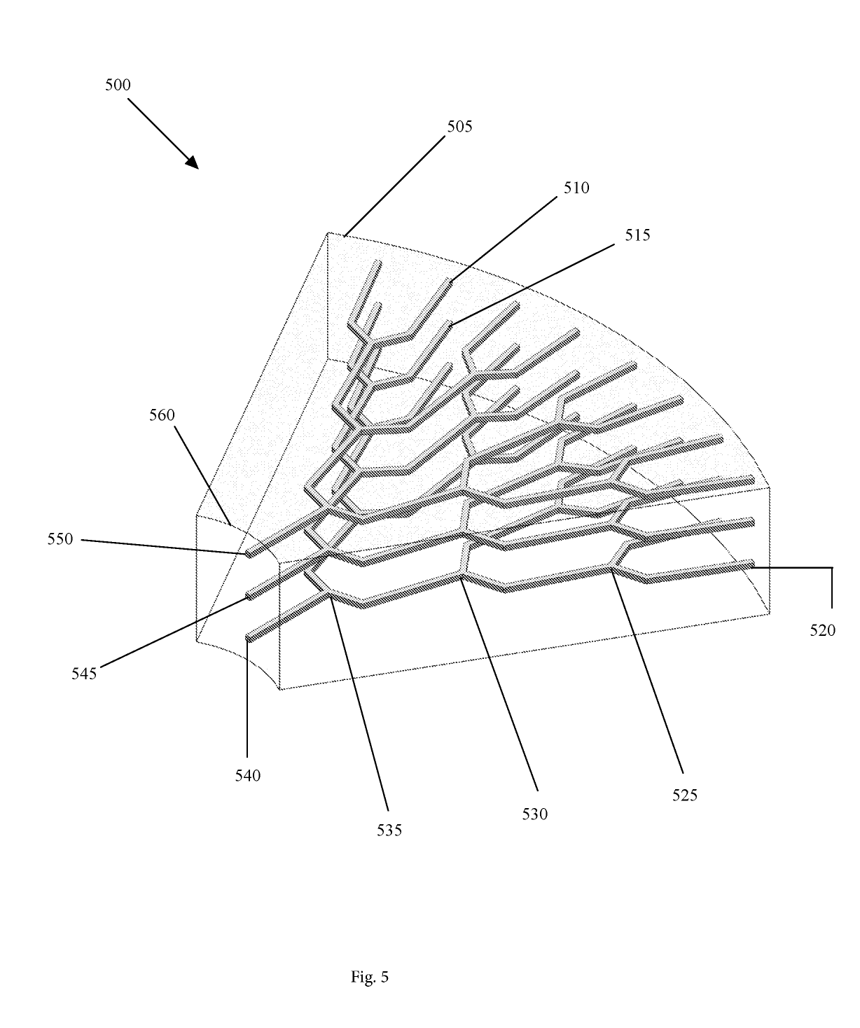

FIG. 5 is an illustration of an individual wedge of a modular microfluidic benthic microbial fuel cell (MBMFC) having three fractal branch channel layers. Microfluidic benthic microbial fuel cell (MBMFC) wedge 500 may include fluid input 540, 545, and 550, fluid output 510, 515, and 520, fractal branch channels 525, 530, and 535, inner inflow arc 560, and outer output arc 505. MBMFC wedge 500 may be one of many units that form one or more stackable layers in (MBMFC) 100 containing anode 125 (as shown in FIG. 1).

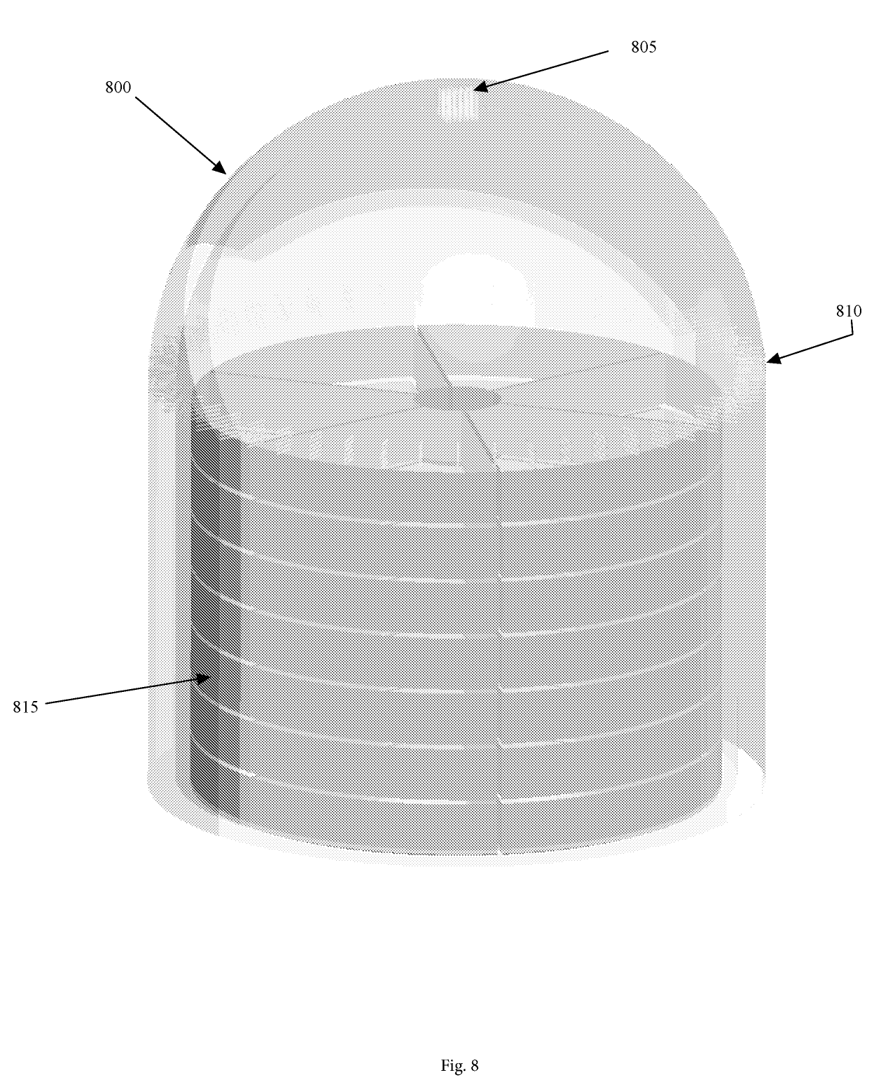

Fluidic input 540, 545, and 550, fluid output 510, 515, and 520, and fractal branch channels 525, 530, and 535, may be fluidically isolated from each other within a single MBMFC wedge 500 in the downstream direction (if the flow is oriented from inside to outside). Fluidic input 540, 545, and 550 may separately accept a fluid flow from a single microbial medium inflow 115 (as shown in FIG. 1). Similarly, fluid output 510, 515, and 520, may separately allow a fluid to flow out to water column 110 (as shown in FIG. 1) or to external enclosure 800 (as shown in FIG. 8). The isolation of fluid input 540, 545, and 550, fluid output 510, 515, and 520, fractal branch channels 525, 530, and 535 may allow a single MBMFC wedge 500 to support a microbial environment in separate fractal branch channels 525, 530, and 535, the microbial environments may be kept independent or combined/connected into a single microbial environment.

Fractal branch channels 525, 530, and 535 may be circular, rectangular, triangular, or oval-shaped, tear drop shaped, or possess other cross-sections designs in MBMFC wedge 500 as MBMFC wedge 500 may be 3D printed. Alternatively, fractal branch channels 525, 530, and 535 may be a lattice of multiple geometric-shaped microorganism environments 320 (as shown in FIGS. 3A and 3B). Alternatively, fractal branch channels 525, 530, and 535 may be a lattice of multiple spicules that form microorganism environments 336 and 337 as shown in FIG. 3B.

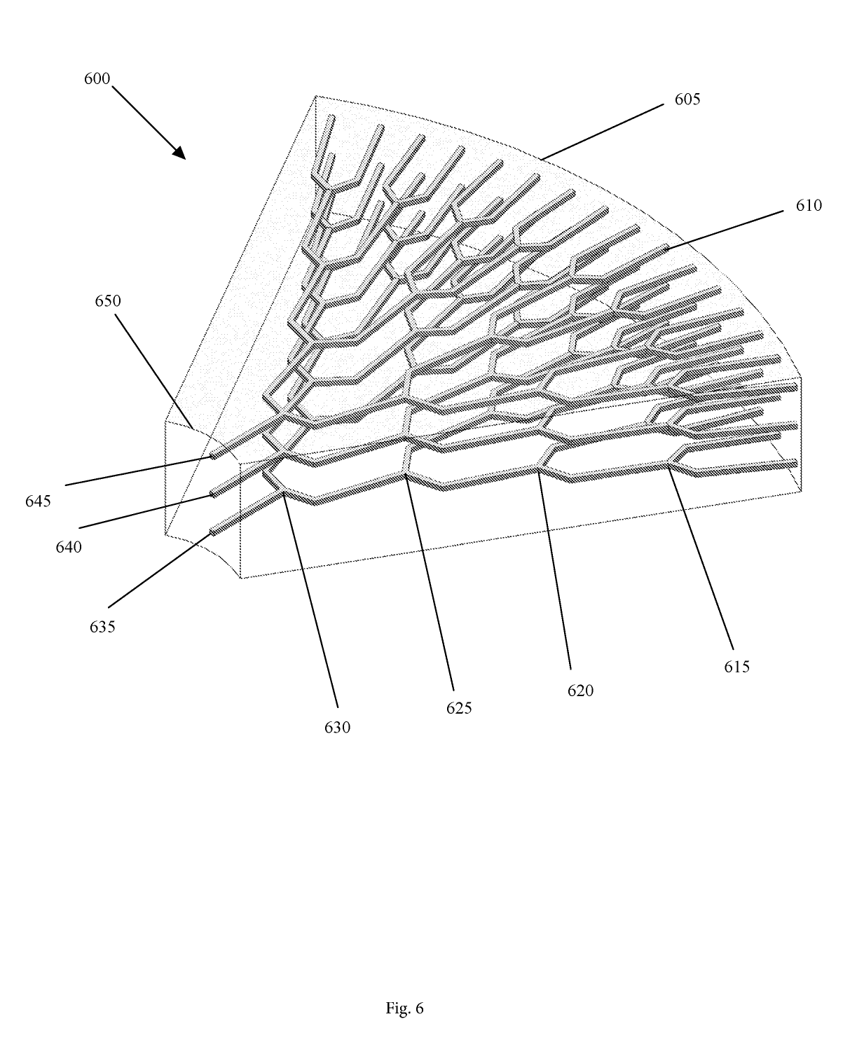

FIG. 6 is an illustration of an individual wedge of a modular microfluidic benthic microbial fuel cell (MBMFC) having three fractal branch channel layers with an increase in fractal channel length. Microfluidic benthic microbial fuel cell (MBMFC) wedge 600 may include fluid input 635, 640, and 645, multiple fluid outputs 610, fractal branch channels 615, 620, 625, and 630, inner inflow arc 650, and outer output arc 605. MBMFC wedge 600 may be one of many units that form one or more stackable layers in (MBMFC) 100 containing anode 125 (as shown in FIG. 1).

Fluid input 635, 640, and 645, multiple fluid output 610, and fractal branch channels 615, 620, 625, and 630 may be fluidically isolated from each other within a single MBMFC wedge 600 in the downstream direction (if the flow is oriented from inside to outside). Fluid input 635, 640, and 645 may separately accept a fluid flow from a single microbial medium inflow 115. Similarly, multiple fluid output 610, may separately allow a fluid to flow out to water column 110. The isolation of input 635, 640, and 645, multiple fluid outputs 610, fractal branch channels 615, 620, 625, and 630 may allow a single MBMFC wedge 600 to continue to support a microbial environment in the separate fractal branch channels 615, 620, 625, and 630, even if one or more channels may be clogged or blocked.

Fractal branch channels 615, 620, 625, and 630 may be circular, rectangular, triangular, oval-shaped, tear drop shaped, or possess other cross-sections designs in MBMFC wedge 600 as MBMFC wedge 600 may be 3D printed. Alternatively, fractal branch channels 615, 620, 625, and 630 may be a lattice of multiple geometric-shaped microorganism environments 320 (as shown in FIG. 3A). Alternatively, fractal branch channels 615, 620, 625, and 630 may be a lattice of multiple spicules that form microorganism environments 336 and 337 (as shown in FIG. 3B).

An alternate embodiment may utilize asymmetric fractal branching, meaning that the fractal branching may increase the number of channels, and the channels do not branch back together (symmetric branching). For example, if clogging occurs during processing/clearing phase or in situ, higher pressure may be applied to only the clogged location by plugging or otherwise blocking off all outlets not directly downstream of the clog then applied pressure at the main inlet. This way the flow may be able to be directed specifically to any single point in the fractal pattern by plugging or otherwise blocking a subset of outlets. A benefit such as but not limited to damaged modules being repaired in the field or by clearing a module of sacrificial support material.

An alternate embodiment, fractal branching may be arranged but should not be limited to a radial or a cartesian. A radial direction arrangement may allow for efficient usage of the available space (both volume for the channels and/or microbial environments and surface area for ports) in a wedge-shaped module, and/or a cylindrical shaped assembly. Using wedge shaped modules, asymmetric fractal branching, radial directed fractal branching, and a cylindrical assembly together may allow for the additional benefits.

Modular MFC (or MBMFC) with MBMFC) wedge 600 or similarly shaped modules (not shown) containing an asymmetric fractal branching patterned channels may provide the benefit of removing all microfluidic to microfluidic (or milli-fluidic to milli-fluidic) connections between individual modules. The microfluidic (or milli-fluidic) to microfluidic (or milli-fluidic) interfaces may be replaced with the outlet channels connecting to the water column on the external surface and the inlet channels connecting to an axial chamber 705. Under this arrangement location/alignment tolerances for assembly, in situ repairs, maintenance, and/or fabrication may be increased leading to a decrease in cost and fabrication/assembly difficulty a more robust assembly.

Due to inherent properties of an asymmetric fractal branching pattern the statistical distribution of the effect of a clog (or similar point failure in the channel flow) may be dependent on position in the fractal pattern, such that, the negative effect would be smaller if the failure occurred downstream of a branching event. Due to the nature of the fractal branching, there may be significantly more channel length after each branching event (due to the increased number of branches), so the chance of a point failure having a larger negative effect may be significantly smaller than the chance of it causing a smaller negative effect due to the difference in effect magnitude from the location in the branching structure and the bias towards locations with smaller effects.

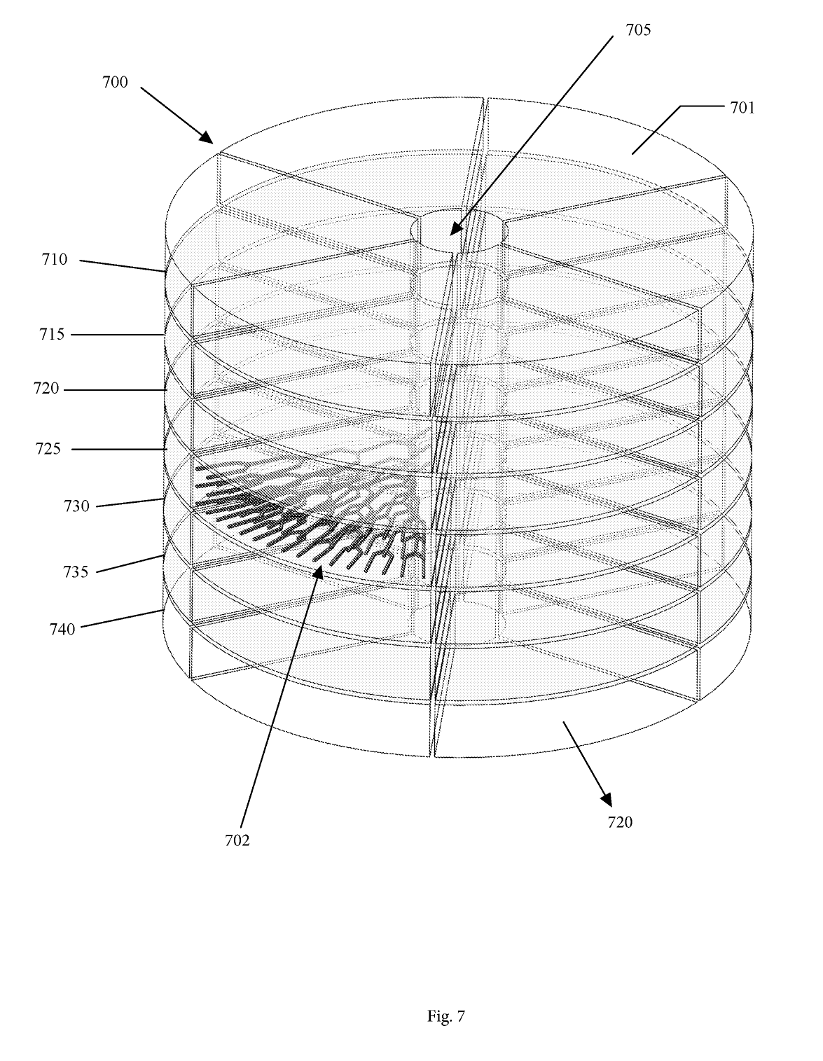

FIG. 7 is one embodiment of a cylindrically assembled modular microfluidic benthic microbial fuel cell (MBMFC) having seven stacked layers and six radially arranged wedge modules per layer. MBMFC assembly 700 should not be limited to any geometric shape. For example, MBMFC assembly 700 may be described here as a cylinder, but may be a pyramid, cube, or any other design.

MBMFC assembly 700 may be formed by stacking multiple rings 710, 715, 720, 725, 730, 735, and 740. Multiple rings 710, 715, 720, 725, 730, 735, and 740 may be formed from multiple MBMFC wedges 701.

Although a common fluid input at the end of axial chamber 705 may be utilized, each ring 710, 715, 720, 725, 730, and 735 may be fluidically isolated from each other downstream of the inlet to the channels (upstream if the direction of flow may be reversed). Additionally, each ring 710, 715, 720, 725, 730, and 735 may preferably have one or more fluid input (not shown) that accept fluid flow from axial chamber 705 and one or more fluid output 702. Each ring 710, 715, 720, 725, 730, and 735 having one or more fluid inputs (not shown) that accept fluid flow from axial chamber 705 and one or more fluid outputs 702 that may improve reliability and resistance to clogs stopping fluid flow.

Stacking multiple fluidically isolated rings 710, 715, 720, 725, 730, 735 to form MBMFC assembly 700 may increase reliability and reduce or potentially eliminate single point failures.

Although FIG. 7 depicts a single MBMFC wedge 710 showing the fractal branch channels, each of multiple MBMFC wedge 701 may include fractal branch channels.

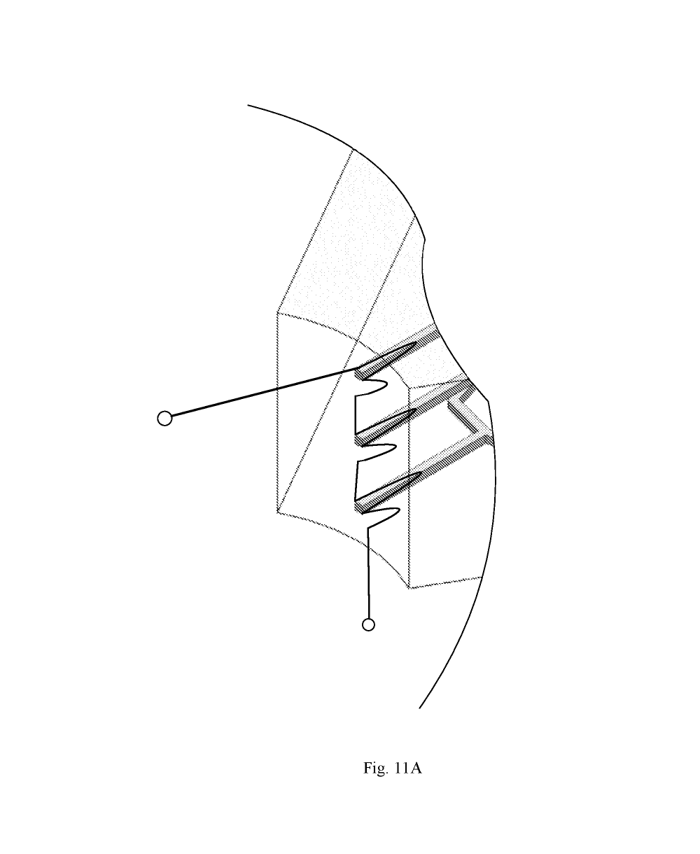

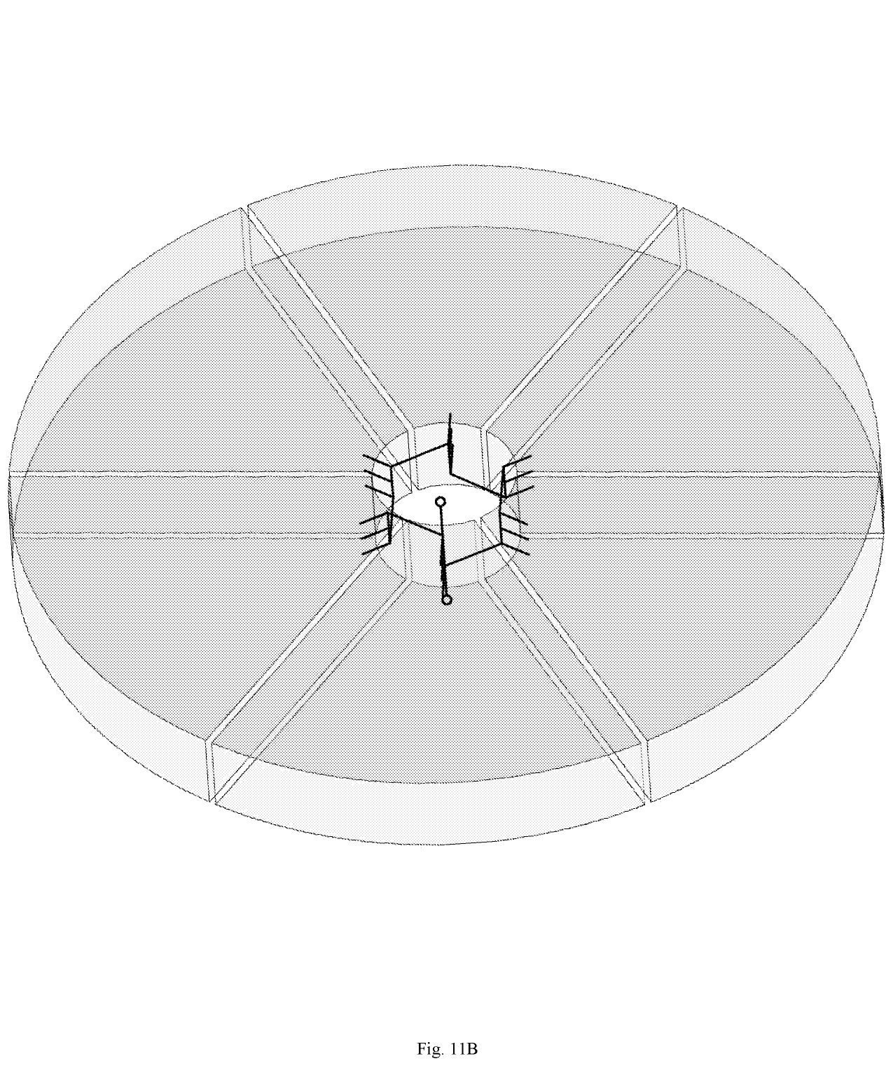

One embodiment MBMFC assembly 700 may include an architecture having a group of wedge modules assembled into a disk with a cavity in the center. Multiple disks may be stacked vertically, and the assembly may take the form of a cylinder with an axial chamber 705 along the central axis. Axial chamber 705 in the center may allow for, but should not be limited to allowing for nutrient or other additive mixing, fluidic connections between each isolated network of fractal branch channels 615, 620, 625, and 630 (as shown in FIG. 6), a location for electrical connections between each anode as shown in FIGS. 11A and 11B, a location to configure a single pump (not shown) to provide flow to all parts of the assembly. This cylindrical geometry may be chosen for its fluidic drag properties, its radial symmetry, its structural properties, its ratio of surface area to volume, its compatibility with wedge/crescent shaped modules, reduced mechanical stresses from the moving water or from any other source, reduced likelihood of fatigue related mechanical failure, performance independent of tides or any other directional bias in external flow, or any other reason.

An alternate embodiment may include multiple individual MBMFC wedges 701 assembled to form multiple rings 710, 715, 720, 725, 730, 735, and 740. The multiple rings 710, 715, 720, 725, 730, 735, and 740 may further be stacked to form a cylinder architecture that forms a complete MBMFC assembly 700.

An Alternate embodiment may include an architecture of MBMFC assembly 700 having a group of wedge modules assembled into a disk with a scale cavity (axial chamber 705) in the center. Multiple disks may be stacked vertically to form a cylinder with a cylindrical hole along the central axis. Axial chamber 705 may allow for, but should not be limited to allowing nutrient or other additive mixing, fluidic connections between each isolated network of fractal branch channels 615, 620, 625, and 630 (as shown in FIG. 6), a location for electrical connections between each anode (as shown in FIG. 11A and FIG. 11B), a location to configure a single pump (not shown) to provide flow to all parts of the assembly, or any other desired use. This external cylindrical geometry may be chosen for its fluidic drag properties, its radial symmetry, its structural properties, its ratio of surface area to volume, its compatibility with wedge/crescent shaped modules, reduced mechanical stresses from the moving water or from any other source, reduced likelihood of fatigue related mechanical failure, performance independent of tides or any other directional bias in external flow, or any other reason. Flow may enter the axial chamber 705 of the MBMFC assembly 700 structure at axial chamber 705 and provide fluid flow to the multiple rings 710, 715, 720, 725, 730, 735, 740 and thereby providing fluid flow to individual MBMFC wedges 701 simultaneously.

A wedge-like geometry at the module level with a cylinder-like geometry at the assembly level may be particularly suited to a marine and/or benthic environment due to increased resilience from, but not limited to, any of the following sources: removal of microfluidic to microfluidic (or milli-fluidic to milli-fluidic) interfaces, radial removal/insertion of wedges into/out of the assembly, the assemblies’ fluidic drag properties, and/or symmetry.

FIG. 8 is one embodiment of a microfluidic benthic microbial fuel cell (MBMFC) having an external enclosure. External enclosure 800 may include filtered inlet 810 and vent outlets 805, and further house MBMFC assembly 815 and a microbial environment. MBMFC assembly 815 should be connected to a cathode in the aerobic, oxygen-rich environment to complete the MFC circuit.

External enclosure 800 may keep nutrients contained for recirculation through MBMFC assembly 815 and the microbial environment. External enclosure 800 may also provide, but should not be limited to providing, recirculation potential, protection from the environment, a more controlled environment, protection from temperature swings, maintaining an anerobic environment while facilitating the necessary exchange of gaseous byproducts, and/or lower effects from differences in the surrounding fluid dynamics such as waves, tides or larger organisms around the area.

Filtered inlet 810 and vent outlets 805 may be used to maintain anerobic environment, provide nutrients, and control escaping oxygen and removal of byproducts to vent outlets 805.

In one embodiment, MBMFC assembly 815, showing stacked rings, can be enclosed in an external enclosure 800 environment with filtered inlet 810 and vent outlet 805 on the top. Filtered inlet 810 may allow for filtered water column to passively or actively enter the enclosure to optimize anaerobic or nutritional conditions while allowing the bacterial byproduct gases to escape vent outlet 805. External enclosure 800 may be used in conjunction with other MFC or MBMFC embodiments.

Alternatively, a nutrient solution can be externally connected to filtered inlet 810 and/or a dedicated port (not shown) to improve power generation caused by the electrogenic bacteria respiration and metabolic cycles or other sources.

In one embodiment modules and/or fragments may be made from watertight materials including materials that may allow small amounts of water to flow. The flow may be controlled or directed though channels.

In an alternate embodiment the material may be used to form the modules/fragments were permeable to the waste products produced by the microorganism allowing for waste products to escape, while impermeable to favorable terminal electron receptors, such as, but not limited to oxygen which may prevent loss of electrons.

An alternate embodiment of External enclosure 800 may include vent outlets 805 for microorganism produced waste products may escape.

An alternate embodiment may utilize a material that may be highly permeable to water (and/or other small particles, objects, and/or microorganisms), allowing the bulk material to act as inlet and/or outlet for the system. This potentially desirable material permeability may be achieved through material properties and/or though intentional design and fabrication of a micro-structure, such as, but not limited to a typical FDM 3D printing infill pattern.

An alternate embodiment may have electronics placed in an underwater, water-proof housing (not shown) to regulate the energy harvested from the MBMFCs.

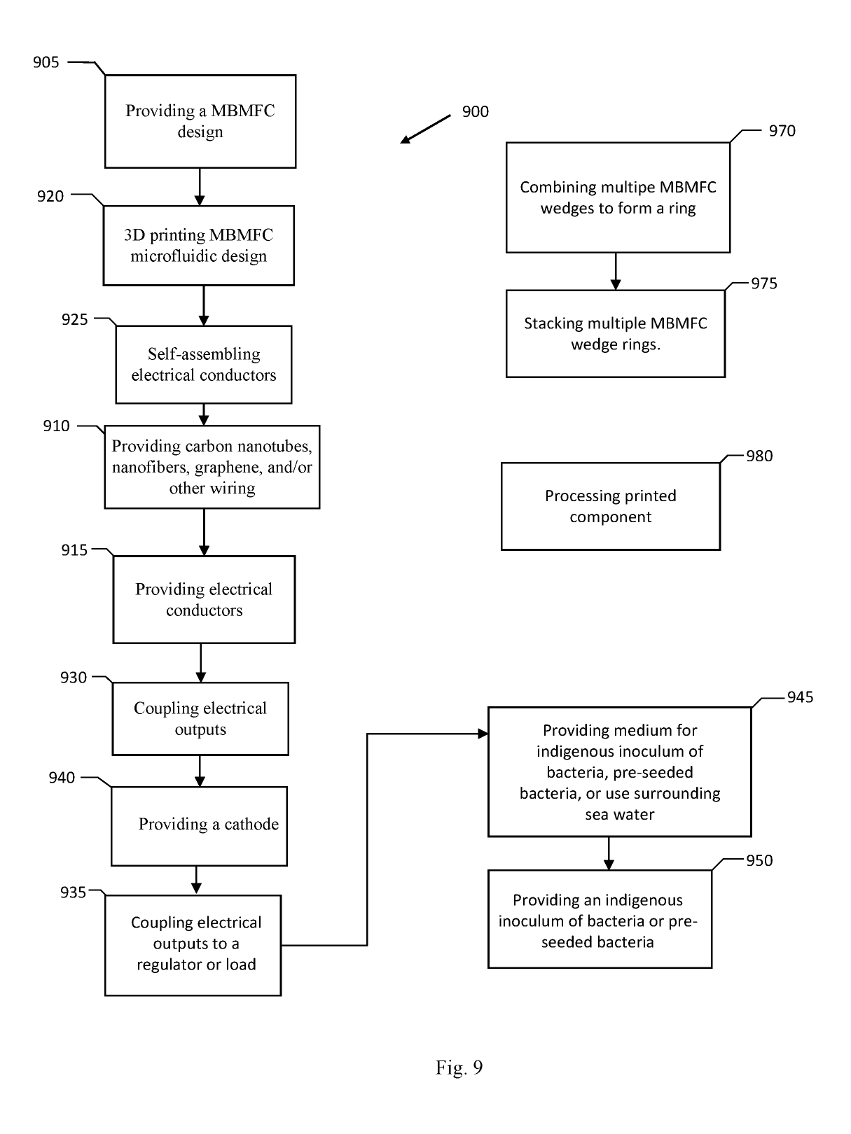

FIG. 9 is a block flow diagram showing the steps to produce a microfluidic benthic microbial fuel cell (MBMFC). Microfluidic benthic microbial fuel cell (MBMFC) 900 may be produced by the process of: providing 905 a MBMFC microfluidic design comprising multiple environmental volumes suitable for microorganisms. A MBMFC design may be a 3D printable design. A 3D printable design may be a digital model that can be converted into a physical object through 3D printing or additive manufacturing generally done layer-by-layer. Providing 910 multiple carbon nanotubes, carbon nanofibers, graphene, and/or other materials. Carbon nanotubes and carbon nanofibers tend to clump together and may require detangling to maintain homogeny. Providing multiple electrical conductors 915, 3D printing 920 multiple layers, a single layer at a time, of the MBMFC microfluidic design by additive techniques or extrusion of carbon nanotubes or other conductive materials. Self-assembling 925, by the multiple electrical conductors, wire conductors electrically coupled to the multiple microbially relevant environment volumes and electrical output. Self-assembling wires may consist of electrically polarized particles that self-assemble in response to an electric field. Self-assembly may also be based on a chemical affinity process, such as hydrophilic/hydrophobic interactions, polymerization, or functional group chemical specificity and subsequent chemical bonding. Self-assembly may also be based on physical parameters such as but not limited to surface tension or surface area to volume ratios. The electrical conductor may be placed after printing or introduced as part of 3D printing directly. Coupling 930 electrical outputs from a microbial environment to a MBMFC wedge electrical output. Coupling 935 electrical outputs of MBMFC wedge to an electrical regulator or a load. Providing a cathode 940, electrically coupled to the electrical regulator or load. Providing a microbial medium 945 such as but not limited to surrounding seawater and providing an indigenous inoculum of bacteria from the local sediment, or pre-seeded bacteria 950.

Additionally, 3D printing 920 may require processing printed component 980. Processing printed component 980 may include but should not be limited to subtractive post-processing. Subtractive post-processing may include but should not be limited to removing some printed material. This may include sanding, polishing, and/or removal of sacrificial material using a variety of methods such as but not limited to tumbling, milling, abrasive blasting, chemical abrasive dipping, heating, vibrating, flushing, pressure washing and/or scraping.

Alternatively, 3D printing 920 may include but should not be limited to the following 3D printing technologies: PolyJet style, SLA style, FDM style printer, and/or Hybrid printing. PolyJet style 3D printing typically utilizes resin and support material. Support material may be removed, which may complete the formation of the microchannels. The microchannels may then wired with carbon nanotubes, carbon nanofibers, graphene and/or other materials or methods, to produce embedded electrical wiring. Then bacteria may be introduced in the channels. SLA style 3D printing utilizes polymers with the channels being filled with an uncured monomer which may be removed later leaving formed channels. A FDM style printer may 3D print non-soluble and fluid-soluble material such as, but not limited to, PVA as support material in a single print, typically by using a pair of extruders to print each material, respectively. The fluid-soluble material may be removed by applying fluidic treatment to open the channels or by flushing channels already partly open. This process may be aided by certain cross-sectional designs, including but not limited to, tear-dropped shaped cross-sections. The channels can also be used to supply nutrients and/or remove gases and byproducts. Hybrid printing may be a printing process that combines multiple printing technologies and converting techniques: such as but not limited to 3D printing resin based conductive composites.

An alternate embodiment may include a solid module/fragment made of conductive material containing fractal branch channels that may be additively manufactured with a fluid soluble material filling part of but not all of the channel’s volume. The conductive material may be but should not be limited to carbon spiked PLA or other conductive and biocompatible material(s). The fluid soluble support material may be but should not be limited to PVA. FDM style 3D printing together with tear-dropped shaped channel cross-sections may benefit this embodiment. By leaving part of the channel volume free of support material (this would be aided by a tear-dropped shaped channel cross-section) the capacity for fluidic throughput may be processed prior to any processing, this allows for fluid to be flushed though the open position of the channel(s) to remove the fluid soluble support material. Under this arrangement the channels may be augmented with additional conductive material (as with other embodiments) if extra conductivity is desired, if further reducing of proximity between electron emission and capture may be desired, and/or for any other reason. In this embodiment, the conductive bulk structure’s surface area can act as an anode (or portion of an anode) while the volume of the structure can interconnect the parts of the anode electrical, thus the bulk structure may be capable of simultaneously serving as any or all of the following: physical structure, microbial environments, capturing anode, anode electrical interconnections.

Alternatively, self-assembling 945 may include constructing by chemical derivatization by active groups using standard coupling reactions, e.g., peptide bonding or opening a vinyl bond (polymerization). Alternatively, self-assembling 945 may also be constructing by deposition by flow and/or evaporation, wherein conductive microfibers (or other conductive materials) mechanically enmesh and become stuck electrostatically, or hydrophilic/hydrophobic interactions. Van der Waals forces, hydrophilic/hydrophobic interactions, and/or other mechanisms may cause conductive microfibers to mechanically enmesh and become stuck. Another embodiment of MBMFC 900 may include combining 970 multiple microfluidic wedges to form a ring. Coupling 930 electrical outputs from microfluidic benthic microbial fuel cell MBMFC wedge electrical outputs to a single electrical output.

Another embodiment of MBMFC 900 may include stacking 975 multiple rings to form an MBMFC assembly with a central chamber (such as but not limited to, axial chamber 705) and electrically coupling the multiple electrical outputs to each other than to the electrical regulator or load.

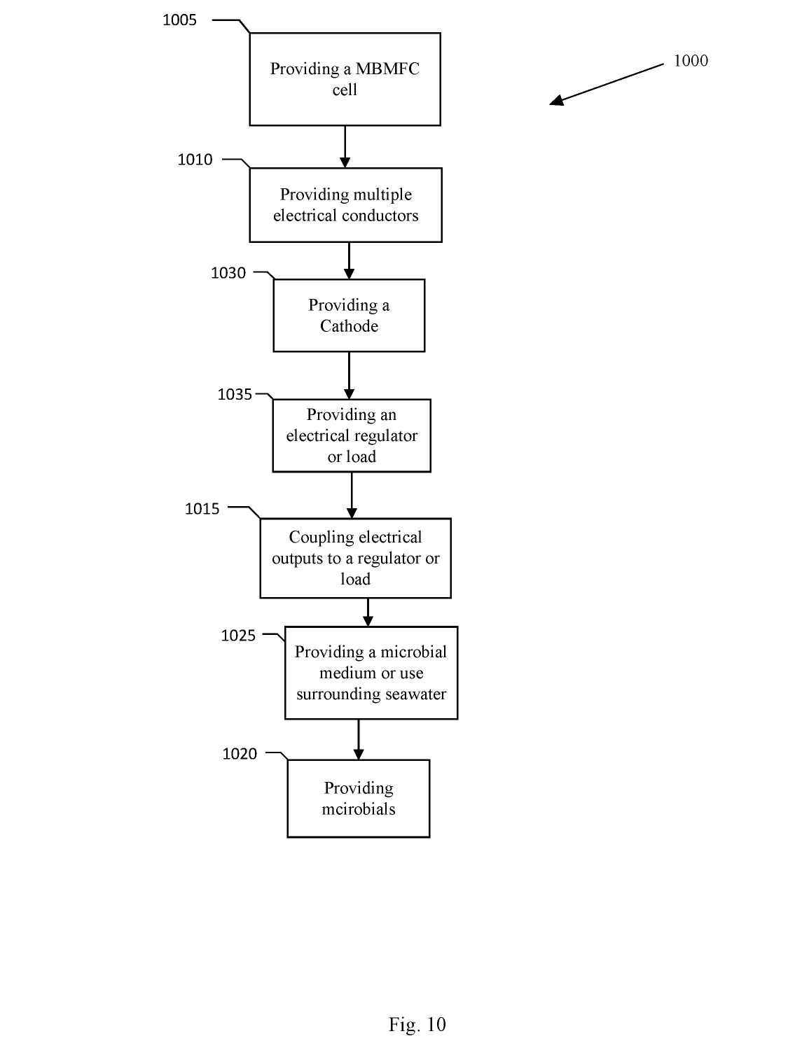

FIG. 10 is a block flow diagram of one method of harnessing electrogenic power by a microfluidic benthic microbial fuel cell (MBMFC). Method of harnessing electrogenic power by a microfluidic benthic microbial fuel cell (MBMFC) 1000 may include the steps of: providing a MBMFC 1005, an MBMFC 500 may include multiple microbial environment volumes, multiple channels, an input, and output, one or more microbial environment volumes, the input, and the output; providing multiple electrical conductors 1010 coupled to the microbial environment volumes and coupled to one or more electrical outputs; coupling 1015 one or more electrical outputs to an electrical regulator or a load; allowing microorganisms to populate 1020 wherein the bacteria release electrons and wherein the released electrons may be collected by the MBMFC; and providing a microbial medium 1025, such as, but not limited to, surrounding seawater wherein the microbial medium may flow through the input, the microbial environmental volumes, the multiple channels, and the output; providing a cathode(s) 1030; and providing an electrical regulator or a load 1035.

FIG. 11A is an illustration of one method of wiring a microfluidic benthic microbial fuel cell (MBMFC). “Voltage reversal” in an MFC (or MBMFC) refers to a situation in which a MFC (or MBMFC) assembly made of individual components, capable of functioning as independent MFCs (or MBMFCs), has an individual component or subset of components that underperforms in terms of power generation. This may have a disproportionate effect on the power generation of the MFC (or MBMFC) assembly. This disproportionality may be a result of the power generated by the other properly functioning individual units in the assembly being diverted to the underperforming unit instead of the output. This may drastically reduce the overall power output of the entire MFC (or MBMFC) assembly. This problem may be overcome by electrically connecting the multiple wedges/modules of the MBMFC such that from an electrical perspective to form a single larger MFC (or MBMFC) made of modules rather than many independent small MFC (or MBMFC) modules. This may be done by interconnecting the anodes in each module to form a single anode assembly that takes a single electrical connection to the cathode(s). Utilizing this design the problem of voltage reversal may be bypassed by creating a single electrical unit comprising multiple physical units instead of having multiple electrical units.

One embodiment showing a method of wiring an MBMFC module is shown in FIG. 11A. In order to connect multiple layers of fractal branch channels a conductor may be configured to make an electrical connection between all anodes. An interconnected wiring network may offer significantly improved performance over a series or parallel arrangement. It is preferable to use a biocompatible conductor, such as, but not limited to, titanium. An electrically conductive biocompatible adhesive, such as but not limited to an epoxy with carbon additives, or other connectors may be used to secure the electrical conductor to the wedge/module or anode. Care should be taken to ensure that adhesive does not block the fractal branch channel inlets. This can be aided by adding curved bends of wire that allow the point where adhesives would be applied far enough away from the channel opening as seen in FIG. 11A.

The concepts disclosed herein may be used in applications independent of MBMFCs or MFCs. For example, a cylindrical assembly of 3D printed wedge modules containing fractal branch channels may be used as a fluidic dynamical design without requiring that it be used to house the anode of an MFC. Unless specifically stated, all embodiments and designs disclosed herein as specific to use with and/or as MBMFCs or MFCs may be intended to be applicable to other use cases as well.

FIG. 11B is an illustration of one method of wiring together individual modules of an MBMFC. As shown in FIG. 11B, a method wiring an MBMFC assembly may also produce multiple electrical pathways from any point on the anode to the cathode, thus further reducing single point failures in the system while simultaneously decreasing the overall resistance. It may also be possible that these connections can serve not only as electrical connections between electrically isolated anodes, but also as part of the anode itself.

Unless otherwise stated, all measurements, values, ratings, positions, magnitudes, sizes, locations, and other specifications that may be set forth in this specification, including in the claims that follow, approximate not exact. They are intended to have a reasonable range that is consistent with the functions to which they relate and with what is customary in the art to which they pertain.

The foregoing description of the preferred embodiment has been presented for the purposes of illustration and description. While multiple embodiments are disclosed, still other embodiments will become apparent to those skilled in the art from the above detailed description. These embodiments are capable of modifications in various obvious aspects, all without departing from the spirit and scope of protection. Accordingly, the detailed description is to be regarded as illustrative in nature and not restrictive. Also, although not explicitly recited, one or more embodiments may be practiced in combination or conjunction with one another. Furthermore, the reference or non-reference to a particular embodiment shall not be interpreted to limit the scope of protection. It is intended that the scope of protection not be limited by this detailed description, but by the claims and the equivalents to the claims that are appended hereto.

Except as stated immediately above, nothing that has been stated or illustrated is intended or should be interpreted to cause a dedication of any component, step, feature, object, benefit, advantage, or equivalent, to the public, regardless of whether it is or is not recited in the claims.

What is claimed is:

1. A microfluidic benthic microbial fuel cell (MBMFC) comprising:
a microbial medium;
a microbial consortia;
multiple microfluidic wedges arranged on a plane to form a tray, each of the microfluidic wedges comprising multiple microbial environment volumes, multiple channels, an input, an output, the multiple microbial environment volumes being in fluidic communications with the multiple channels, the input, and the output; and
multiple electrical conductors coupled to the multiple microbial environment volumes and coupled to an electrical output.

2. The MBMFC of claim 1, wherein the microbial medium flows through the input, the multiple channels, the multiple microbial environment volumes, and the output;
wherein the microbial consortia live in the multiple microbial environment volumes; and
wherein the multiple microbial environment volumes are configured to capture electrons.

3. The MBMFC of claim 1, wherein one or more dimensions of the multiple microbial environment volume range from 1 micro-meter cubed (µm³⁾ to 10 cubic meters (m³⁾; and
the multiple microbial environment volumes receive a nutrient import and waste export through the flow of the microbial medium.

4. The MBMFC of claim 1, wherein the multiple microbial environment volumes are configured to retain a biofilm layer comprising the microbial consortia and an extracellular polymeric substance.

5. The MBMFC of claim 1, further comprising an electrical regulator or a load;
the electrical regulator or load being electrically coupled to the output of the one or more microfluidic wedges.

6. The MBMFC of claim 5, further comprising a cathode electrically coupled to the electrical regulator or load.

7. The MBMFC of claim 5, further comprising multiple microfluidic trays stacked to form an anode; and
the multiple electrical conductor outputs being coupled to the electrical regulator or load.

8. The MBMFC of claim 1, wherein the multiple microbial environment volumes, the multiple channels, the input, the output are made from carbon or a conductive matrix.

9. The MBMFC of claim 8, wherein the carbon is carbon nanotubes.

10. The MBMFC of claim 8 wherein the carbon is graphene or carbon nanofibers.

11. The MBMFC of claim 8, wherein the carbon is carbon nanotubes, carbon nanofibers, and graphene.

12. The MBMFC of claim 1, wherein the multiple electrical conductors are biocompatible conductive conductors selected from the group consisting of: stainless steel, chrome, titanium, tantalum, niobium, and gold.

13. A microfluidic benthic microbial fuel cell (MBMFC) produced by the process of:
providing a MBMFC microfluidic design comprising multiple microbial environment volumes, multiple channels, an input, an output, the multiple microbial environment volumes being in fluidic communications with the multiple channels, the input, and the output;
providing multiple carbon nanofibers;
providing multiple electrical conductors;
3-dimensional printing multiple layers, a single layer at a time, of the MBMFC microfluidic design by extrusion of the multiple carbon nanofibers;
self-assembling, by the multiple electrical conductors, one or more wire conductors electrically coupled to the multiple microbial environment volumes and an electrical output; and
coupling one or more electrical outputs to an electrical regulator or a load.

14. The MBMFC produced by the process of claim 13, further comprising providing a microbial medium and providing a microbial consortia, wherein the microbial consortia is a bacteria.

15. The MBMFC produced by the process of claim 13, wherein one or more dimensions of the multiple microbial environment volume range from 1 cubic micro-meter (µm³) to 10 cubic meters (m³); and
wherein the multiple microbial environment volumes receive a nutrient import through the flow of the microbial medium.

16. The MBMFC produced by the process of claim 14, wherein the multiple microbial environment volumes are configured to export a waste through the flow of the microbial medium.

17. The MBMFC produced by the process of claim 13, further comprising providing a cathode electrically coupled to the electrical regulator or load.

18. The MBMFC produced by the process of claim 13, further comprising:
combing multiple microfluidic wedges to form a tray;
stacking multiple trays to form an anode; and
electrically coupling multiple electrical outputs of the multiple microfluidic wedges to the electrical regulator or load.

19. A method of harnessing electrogenic power by a microfluidic benthic microbial fuel cell (MBMFCs) comprising:
providing a MBMFC cell comprising multiple microbial environment volumes, multiple channels, an input, an output, the multiple microbial environment volumes being in fluidic communications with the multiple channels, the input, and the output;
providing multiple electrical conductors coupled to the multiple microbial environment volumes and coupled to one or more electrical outputs;
coupling the one or more electrical outputs to a tray output;
coupling one or more tray outputs to an electrical regulator or a load;
providing a microbial consortia, wherein the microbial consortia release one or more electrons and wherein the one or more released electrons are collected by the MBMFC cell; and
providing a microbial medium, wherein the microbial medium flows through the input, the multiple microbial environment volumes, the multiple channels, and the output.

20. The method of harnessing electrogenic power by a MBMFCs of claim 19, further comprising providing a cathode; and an electrical regulator or a load.
